(19) United States
(12) Patent Application Publication
(10) Pub. No.: US 2019/0015343 A1
(43) Pub. Date: Jan. 17, 2019

(54) COMBINATION OF DRUGS HAVING DIFFERENT POTENCY

(71) Applicant: GRÜNENTHAL GMBH, Aachen (DE)

(72) Inventors: Carmen STOMBERG, Korschenbroich (DE); Klaus WENING, Köln (DE); Marcel HAUPTS, Stolberg (DE)

(73) Assignee: GRÜNENTHAL GMBH, Aachen (DE)

(21) Appl. No.: 16/134,059

(22) Filed: Sep. 18, 2018

Related U.S. Application Data
(63) Continuation of application No. PCT/EP2017/056417, filed on Mar. 17, 2017.

(30) Foreign Application Priority Data
Mar. 18, 2016 (EP) 16161192.6

(51) Int. Cl. A61K 9/20; A61K 9/24; A61K 31/135

(52) U.S. Cl. CPC A61K 9/2095; A61K 9/209; B33Y 80/00; A61K 9/204; A61K 9/2054; A61K 31/135

(57) ABSTRACT

The invention relates to a three-dimensionally printed pharmaceutical dosage form comprising a first pharmacologically active ingredient and a second pharmacologically active ingredient; wherein the relative weight ratio of the first pharmacologically active ingredient to the second pharmacologically active ingredient in the pharmaceutical dosage form is within the range of from 10,000:1 to 1:1. The invention also relates to a process for the preparation of such pharmaceutical dosage form by three-dimensional printing, preferably by fused deposition modeling.

This application is a continuation of International Patent Application No. PCT/EP2017/056417, filed Mar. 17, 2017, which claims priority of European Patent Application No. 16161192.6, filed Mar. 18, 2016, the contents of which patent applications are incorporated herein by reference.

The invention relates to a three-dimensionally printed pharmaceutical dosage form comprising a first pharmacologically active ingredient and a second pharmacologically active ingredient; wherein the relative weight ratio of the first pharmacologically active ingredient to the second pharmacologically active ingredient in the pharmaceutical dosage form is within the range of from 10,000:1 to 20:1. The invention also relates to a process for the preparation of such pharmaceutical dosage form by three-dimensional printing, preferably by fused deposition modeling.

Many drugs that are suitable and advantageously useful for combination therapy have highly diverse specific potency, i.e. one drug of the combination needs to be administered at a comparatively low dose, e.g. of a few μg only, whereas the other drug of the combination needs to be administered at a comparatively high dose, e.g. of several hundred mg.

When pharmaceutical dosage forms containing only a low dose of drug are fabricated with conventional manufacturing techniques, it is a particular challenge to ensure that each pharmaceutical dosage form of a batch has the same drug content (content uniformity). In a pharmaceutical dosage form containing a low content of drug, most of the pharmaceutical dosage form will consist of other excipients than the drug. When fabricating pharmaceutical dosage forms with conventional techniques such as e.g. direct compression or extrusion techniques, first all excipients and the drug are mixed and the resulting mixture is then partitioned and shaped to pharmaceutical dosage forms. Thus, when pharmaceutical dosage forms containing a low content of drug are fabricated with conventional techniques a small amount of drug has to be distributed homogeneously in a large mass of other excipients in order to achieve content uniformity.

Furthermore, many drugs that are suitable and advantageously useful for combination therapy need to be released from pharmaceutical dosage forms according to different release profiles. For example, combination of drugs are known where one drug of the combination needs to be released immediately, whereas the other drug of the combination needs to be released in a prolonged or delayed manner. In some instances, it is even desirable that release of one drug of the combination commences only after essentially the total amount of the other drug of the combination has been released already.

Conventional processes for the preparation of pharmaceutical dosage forms satisfying the above requirements are comparatively laborious and expensive. Precise dosing and ensuring content uniformity require specific measures that may be complicated and that may require sophisticated equipment.

WO 98/36738 relates to a rapidly dispersing pharmaceutical dosage form that can incorporate two or more medicaments and can be obtained by three-dimensional printing.

WO 2014/075185 discloses an antibiotic-eluting article for implantation into a mammalian subject produced by an additive manufacturing process wherein a polymeric material is concurrently deposited with a selected antibiotic.

Shaban, A. K., et al.; “3D printing of tablets containing multiple drugs with defined release profiles”, Int. J. Pharm., 494, (2015), p. 643-650, discloses a 3D-printed, complex multi-drug tablet with each drug released with a different profile, wherein the ingredients were separated in compartments.

Shaban, A. K., et al.; “3D printing of five-in-one dose combination polypill with defined immediate and sustained release profiles”, J. of Controlled Release, 217, (2015), p. 308-314, discloses 3D-printing to manufacture a multi-active solid pharmaceutical dosage form containing five compartmentalized drugs with two independently controlled and well-defined release profiles.

The conventional pharmaceutical dosage forms are not satisfactory in every respect and there is a demand for pharmaceutical dosage forms that comprise combinations of at least two drugs and that can be easily prepared. The pharmaceutical dosage forms should allow for formulation of drugs having highly diverse specific potency such that one drug is typically present in excess compared to the other drug and/or should provide different release profiles for the individual drugs.

It is an object of the invention to provide pharmaceutical dosage forms having advantages compared to the prior art.

This object has been achieved by the subject-matter of the patent claims.

It has been surprisingly found that by three-dimensional printing technology the drawbacks of the prior art can be overcome, i.e. that pharmaceutical dosage forms can easily be provided comprising drugs which have highly diverse specific potency and/or specific efficacy and/or are released according to different release profiles. Furthermore, it has been surprisingly found that in the course of preparing polypharmaceuticals, three-dimensional printing technology may reduce the number of process steps and thus facilitates preparation.

A first aspect of the invention relates to a process for the preparation of a pharmaceutical dosage form, which is preferably for oral administration, comprising
a first pharmacologically active ingredient;
a second pharmacologically active ingredient;
optionally, a third pharmacologically active ingredient; and
optionally, a fourth pharmacologically active ingredient;
   wherein the relative weight ratio of the first pharmacologically active ingredient to the second pharmacologically active ingredient in the pharmaceutical dosage form is within the range of from 10,000:1 to 1:1, preferably 10,000:1 to 20:1;
   the process comprising the steps of
(a) providing a first three-dimensionally printable pharmaceutical composition comprising the first pharmacologically active ingredient;
(b) providing a second three-dimensionally printable pharmaceutical composition comprising the second pharmacologically active ingredient;
(b′) optionally, providing a third three-dimensionally printable pharmaceutical composition comprising the third pharmacologically active ingredient;
(b″) optionally, providing a fourth three-dimensionally printable pharmaceutical composition comprising the fourth pharmacologically active ingredient; and
(c) three-dimensionally printing the pharmaceutical dosage form from the first pharmaceutical composition and the second pharmaceutical composition.

It has been surprisingly found that three-dimensional printing technology is particularly useful for combining two different pharmacologically active ingredients having substantially different specific efficacy. There is no other technology that allows for precisely dosing two different pharmacologically active ingredients having substantially different specific efficacy in a single step of manufacture (i.e. a single three-dimensional printing step).

The pharmaceutical composition comprising the pharmacologically active ingredient having the higher specific efficacy can be provided, e.g. by extruding a filament. In such filament, the low dosed pharmacologically active ingredient is homogeneously distributed and provided in a form that may be precisely printed and thus dosed. For example, when employing a printing nozzle having a diameter of 0.1 mm, the printing resolution is about 0.1 mm³ such that dosing can be achieved very precisely. This cannot be achieved by any conventional technology used for the preparation of pharmaceutical dosage forms, e.g. direct compression.

Further, computer aided printing technology also ensures content uniformity in the course of the preparation of various pharmaceutical dosage forms which cannot be achieved by any conventional technology either when two different pharmacologically active ingredients having substantially different specific efficacy are to be formulated.

In conventional tabletting technology, content uniformity for pharmacologically active ingredients that are contained at low doses, i.e. that have a high efficacy, is typically achieved by blending said pharmacologically active ingredients with a large excess of excipients and manufacturing the pharmaceutical dosage form from such blend. The diluting effect of the excipients in the blend facilitates accurate and precise adjustment of the final dose of the pharmacologically active ingredient in the pharmaceutical dosage form. The pharmaceutical dosage form according to the invention, however, is prepared by three-dimensional printing technology and said two different pharmacologically active ingredients may be comprised in two different filaments. In contrast to conventional tabletting technology, the two filaments may be precisely deposited (e.g. in form of voxels) at predetermined different locations within the pharmaceutical dosage form without requiring homogeneous mixture or dilution by a large excess of excipients. Therefore, when fabricating a pharmaceutical dosage form comprising an active ingredient in a low dose, in contrast to conventional manufacturing techniques it is not mandatory, but can still be advantageous, to distribute a small amount of said active ingredient homogeneously in a large excess of other excipients.

According to the invention, content uniformity can be achieved by independently adjusting the concentration of each pharmacologically active ingredient in each filament, and also by adjusting the amount of each filament which is deposited in the course of printing thereby yielding the pharmaceutical dosage form. For example, on the one hand, when a filament comprises a pharmacologically active ingredient in a comparatively high concentration, a comparatively low amount of this filament needs to be deposited in the course of printing in order to accomplish the desired overall content of this active ingredient in the pharmaceutical dosage form. On the other hand, when a filament comprises said active ingredient in a comparatively low concentration, a comparatively high amount of this filament needs to be deposited in the course of printing in order to accomplish the desired overall content of active ingredient in the pharmaceutical dosage form.

Depending upon the accuracy (e.g. resolution) of the printing device, the pharmacologically active ingredient with the lower dose, i.e. with the higher efficacy, is preferably contained in a filament at a concentration which is lower than the concentration of the other pharmacologically active ingredient with the higher dose, i.e. with the lower efficacy, in the other filament. For example, when the desired total dose of the pharmacologically active ingredient with the lower dose, i.e. with the higher efficacy, is e.g. 5.0 μg, it can be advantageous to provide said pharmacologically active ingredient in a filament wherein it is relatively diluted by a comparatively high content of excipients. Otherwise, minor deviations in the amount of deposited material during printing would result in substantial deviations of dose thereby deteriorating content uniformity. Thus, according to the invention the conventional diluting effect of excipients may specifically be applied to the filament containing the pharmacologically active ingredient with the lower dose thereby further improving accuracy of dosing and hence content uniformity.

The first pharmacologically active ingredient and the second pharmacologically active ingredient that are contained in the pharmaceutical dosage according to the invention form differ from one another, i.e. are not identical, but are not particularly limited.

In this regard, for the purpose of the specification, different derivatives of one and the same drug, e.g. different salts, are to be regarded as different pharmacologically active ingredients. The pharmaceutical dosage form may contain the first pharmacologically active ingredient in combination with the second pharmacologically active ingredient as the only pharmacologically active ingredients. It is also possible that the pharmaceutical dosage form contains additional pharmacologically active ingredients, e.g. a third pharmacologically active ingredient.

Preferably, the first pharmacologically active ingredient and the second pharmacologically active ingredient have a different specific potency. More preferably, the second pharmacologically active ingredient is more potent in terms of specific potency than the first pharmacologically active ingredient.

Preferably, the first pharmacologically active ingredient and the second pharmacologically active ingredient have a different specific efficacy. More preferably, the second pharmacologically active ingredient is more efficient in terms of specific efficacy than the first pharmacologically active ingredient.

Potency and efficacy describe different properties of a pharmacologically active ingredient. The specific potency of a pharmacologically active ingredient refers to the concentration of the active ingredient that is required to achieve a given effect. It is usually expressed by the EC₅₀-value which is the concentration of active ingredient that produces 50% of the maximum possible response to the active ingredient. Thus, the most potent active ingredient is the one with the lowest EC₅₀-value. The specific efficacy of a pharmacologically active ingredient refers to the maximum level of response which can be elicited by the active ingredient regardless of the dose. For example, when an active ingredient has a high specific potency and a high specific efficacy, only a low concentration is necessary to achieve a high level of response. When an active ingredient has a low specific potency and a high specific efficacy, a high concentration is necessary to achieve a high level of response.

In a preferred embodiment, the first pharmacologically active ingredient is for the treatment of diseases or disorders of the nervous system [ATC code N]; more preferably an analgesic [ATC code N02], or an antiepileptic [ATC code N03], or a psychoanaleptic [ATC code N06]; most preferably an opioid [ATC code N02A], an antiepileptic [ATC code N03A], or a psychostimulant [ATC code N06B].

In a preferred embodiment, the second pharmacologically active ingredient independently is for the treatment of diseases or disorders of the nervous system [ATC code N]; more preferably an analgesic [ATC code N02], or an antiepileptic [ATC code N03], or a psychoanaleptic [ATC code N06]; most preferably an opioid [ATC code N02A], an antiepileptic [ATC code N03A], or a psychostimulant [ATC code N06B].

In a preferred embodiment, the pharmaceutical dosage form additionally comprises a third pharmacologically active ingredient that may be contained in the first pharmaceutical composition, in the second pharmaceutical composition, in a third pharmaceutical composition, or may be divided and distributed over different pharmaceutical compositions. In a preferred embodiment, the third pharmacologically active ingredient independently is for the treatment of diseases or disorders of the nervous system [ATC code N]; more preferably an analgesic [ATC code N02], or an antiepileptic [ATC code N03], or a psychoanaleptic [ATC code N06]; most preferably an opioid [ATC code N02A], an antiepileptic [ATC code N03A], or a psychostimulant [ATC code N06B].

In a preferred embodiment, the pharmaceutical dosage form additionally comprises a fourth pharmacologically active ingredient that may be contained in the first pharmaceutical composition, in the second pharmaceutical composition, in the third pharmaceutical composition (if any), in a fourth pharmaceutical composition, or may be divided and distributed over different pharmaceutical compositions. In a preferred embodiment, the fourth pharmacologically active ingredient independently is for the treatment of diseases or disorders of the nervous system [ATC code N]; more preferably an analgesic [ATC code N02], or an antiepileptic [ATC code N03], or a psychoanaleptic [ATC code N06]; most preferably an opioid [ATC code N02A], an antiepileptic [ATC code N03A], or a psychostimulant [ATC code N06B].

The relative weight ratio of the first pharmacologically active ingredient to the second pharmacologically active ingredient in the pharmaceutical dosage form is within the range of from 10,000:1 to 1:1, preferably 10,000:1 to 20:1. Unless expressly stated otherwise, the weight is expressed in terms of the equivalent weight with respect to the non-salt form of the first and second pharmacologically active ingredient.

Preferably, the relative weight ratio of the first pharmacologically active ingredient to the second pharmacologically active ingredient in the pharmaceutical dosage form is within the range of from 10,000:1 to 2:1, or 10,000:1 to 5:1, or 10,000:1 to 10:1, or 10,000:1 to 20:1, or 10,000:1 to 25:1, or 10,000:1 to 50:1, or 10,000:1 to 100:1, or 10,000:1 to 150:1, or 10,000:1 to 200:1, or 10,000:1 to 250:1; more preferably 5000:1 to 2:1, or 5000:1 to 5:1, or 5000:1 to 10:1, or 5000:1 to 20:1, or 5000:1 to 25:1, or 5000:1 to 50:1, or 5000:1 to 100:1, or 5000:1 to 150:1, or 5000:1 to 200:1, or 5000:1 to 250:1; still more preferably 2500:1 to 2:1, or 2500:1 to 5:1, or 2500:1 to 10:1, or 2500:1 to 20:1, or 2500:1 to 25:1, or 2500:1 to 50:1, or 2500:1 to 100:1, or 2500:1 to 150:1, or 2500:1 to 200:1, or 2500:1 to 250:1; yet more preferably 1000:1 to 2:1, or 1000:1 to 5:1, or 1000:1 to 10:1, or 1000:1 to 20:1, or 1000:1 to 25:1, or 1000:1 to 50:1, or 1000:1 to 100:1, or 1000:1 to 150:1, or 1000:1 to 200:1, or 1000:1 to 250:1; even more preferably 900:1 to 2:1, or 900:1 to 5:1, or 900:1 to 10:1, or 900:1 to 20:1, or 900:1 to 25:1, or 900:1 to 50:1, or 900:1 to 100:1, or 900:1 to 150:1, or 900:1 to 200:1, or 900:1 to 250:1; most preferably 800:1 to 2:1, or 800:1 to 5:1, or 800:1 to 10:1, or 800:1 to 20:1, or 800:1 to 25:1, or 800:1 to 50:1, or 800:1 to 100:1, or 800:1 to 150:1, or 800:1 to 200:1, or 800:1 to 250:1; and in particular 750:1 to 2:1, or 750:1 to 5:1, or 750:1 to 10:1, or 750:1 to 20:1, or 750:1 to 25:1, or 750:1 to 50:1, or 750:1 to 100:1, or 750:1 to 150:1, or 750:1 to 200:1, or 750:1 to 250:1.

Preferably, step (c) involves fused deposition modeling.

Machines for fused deposition modeling (FDM) are commercially available. The machines may dispense multiple materials to achieve different goals: For example, one material may be used to build up the pharmaceutical dosage form and another material may be used to build up a soluble support structure.

In FDM the pharmaceutical dosage form is produced by extruding small flattened strings of molten material to form layers as the material hardens immediately after extrusion from the nozzle. A thermoplastic filament is unwound from a coil and supplies material to an extrusion nozzle which can turn the flow on and off. A worm-drive may push the filament into the nozzle at a controlled rate. The nozzle is heated to melt the material. The thermoplastic material is heated above its glass transition temperature and is then deposited by an extrusion die. The nozzle can be moved in both horizontal and vertical directions by a numerically controlled mechanism. The nozzle follows a tool-path controlled by a computer-aided manufacturing (CAM) software package, and the pharmaceutical dosage form is built from the bottom up, one layer at a time. Stepper motors or servo motors are typically employed to move the extrusion die. The mechanism used is often an X-Y-Z rectilinear design, although other mechanical designs such as deltabot have been employed. Myriad materials are commercially available, such as polylactic acid (PLA), polyamide (PA), among many others (see Ursan et al., J Am Pharm Assoc (2003) 2013, 53(2), 136.44; Prasad et al., Drug Dev Ind Pharm 2015, 1-13).

Pharmaceutical compositions that are suitable to be employed in the three-dimensional printing step according to the invention, preferably in fused deposition modeling, are preferably identical to or at least similar with pharmaceutical compositions that have been known to be suitable for processing by conventional hot melt extrusion technology. Fused deposition modeling has many similarities with conventional hot melt extrusion.

According to a preferred embodiment of the process according to the invention, the first pharmaceutical composition comprises at least 1.0 wt.-%, or at least 2.0 wt.-%, or at least 3.0 wt.-%, or at least 4.0 wt.-%, or at least 5.0 wt.-%; more preferably at least 6.0 wt.-%, or at least 7.0 wt.-%, or at least 8.0 wt.-%, or at least 9.0 wt.-%, or at least 10 wt.-%; still more preferably at least 11 wt.-%, or at least 12 wt.-%, or at least 13 wt.-%, or at least 14 wt.-%, or at least 15 wt.-%; yet more preferably at least 16 wt.-%, or at least 17 wt.-%, or at least 18 wt.-%, or at least 19 wt.-%, or at least 20 wt.-%; even more preferably at least 21 wt.-%, or at least 22 wt.-%, or at least 23 wt.-%, or at least 24 wt.-%, or at least 25 wt.-%; most preferably at least 26 wt.-%, or at least 27 wt.-%, or at least 28 wt.-%, or at least 29 wt.-%, or at least 30 wt.-%; and in particular at least 31 wt.-%, or at least 32 wt.-%, or at least 33 wt.-%, or at least 34 wt.-%, or at least 35 wt.-%; of the first pharmacologically active ingredient, relative to the total weight of the first pharmaceutical composition.

According to a preferred embodiment of the process according to the invention, the second pharmaceutical composition comprises at most 25 wt.-%, or at most 24 wt.-%, or at most 23 wt.-%, or at most 22 wt.-%, or at most 21 wt-%; more preferably at most 20 wt.-%, or at most 19 wt.-%, or at most 18 wt.-%, or at most 17 wt.-%, or at most 16 wt-%; still more preferably at most 15 wt.-%, or at most 14 wt.-%, or at most 13 wt.-%, or at most 12 wt.-%, or at most 11 wt-%; yet more preferably at most 10 wt.-%, or at most 9.0 wt.-%, or at most 8.0 wt.-%, or at most 7.0 wt.-%, or at most 6.0 wt-%; even more preferably at most 5.0 wt.-%, or at most 4.0 wt.-%, or at most 3.0 wt.-%, or at most 2.0 wt.-%, or at most 1.0 wt-%; most preferably at most 0.9 wt.-%, or at most 0.8 wt.-%, or at most 0.7 wt.-%, or at most 0.6 wt-%; and in particular at most 0.5 wt.-%, or at most 0.4 wt.-%, or at most 0.3 wt.-%, or at most 0.2 wt.-%, or at most 0.1 wt-%; of the second pharmacologically active ingredient, relative to the total weight of the second pharmaceutical composition.

According to another preferred embodiment of the process according to the invention
(i) the first pharmaceutical composition comprises the total amount of the first pharmacologically active ingredient; and/or
(ii) the second pharmaceutical composition comprises the total amount of the second pharmacologically active ingredient.

Preferably, the first pharmaceutical composition comprises the first pharmacologically active ingredient in a concentration that differs from the concentration of the second pharmacologically active ingredient in the second pharmaceutical composition.

Preferably, the concentration of the first pharmacologically active ingredient in the first pharmaceutical composition is at least twice as high, or at least 3 times higher, or at least 4 times higher, or at least 5 times higher, or at least 6 times higher, or at least 7 times higher, or at least 8 times higher, or at least 9 times higher, or at least 10 times higher, or at least 12 times higher, or at least 14 times higher, or at least 16 times higher, or at least 18 times higher, or at least 20 times higher, or at least 22 times higher, or at least 24 times higher, or at least 26 times higher, or at least 28 times higher, or at least 30 times higher, in each case than the concentration of the second pharmacologically active ingredient in the second pharmaceutical composition.

Preferably, the pharmaceutical dosage form is prepared by three-dimensionally printing at least two different pharmaceutical compositions, namely the first pharmaceutical composition and the second pharmaceutical composition, which preferably are provided each in form of filaments useful for fused deposition modeling. In a preferred embodiment, the pharmaceutical dosage form is prepared by additionally three-dimensionally printing another pharmaceutical composition that preferably does not contain pharmacologically active ingredients.

Preferably, the printing step (c) involves fused deposition modeling and the first pharmaceutical composition and/or the second pharmaceutical composition are deposited through an extrusion die having a diameter of at most 0.1 mm, or of at most 0.2 mm, or of at most 0.3 mm, or of at most 0.4 mm, or of at most 0.5 mm, or of at most 0.6 mm, or of at most 0.7 mm, or of at most 0.8 mm, or of at most 0.9 mm, more preferred of at most 1.0 mm, or of at most 1.1 mm, or of at most 1.2 mm, or of at most 1.3 mm, or of at most 1.4 mm, or of at most 1.5 mm, or of at most 1.6 mm, or of at most 1.7 mm, or of at most 1.8 mm, or of at most 1.9 mm, or of at most 2.0 mm, or of at most 2.1 mm, or of at most 2.2 mm, or of at most 2.3 mm, or of at most 2.4 mm, or of at most 2.5 mm, or of at most 2.6 mm, or of at most 2.7 mm, or of at most 2.8 mm, or of at most 2.9 mm, or of at most 3.0 mm, or of at most 3.1 mm, or of at most 3.2 mm, or of at most 3.3 mm, or of at most 3.4 mm, or of at most 3.5 mm, or of at most 3.6 mm, or of at most 3.7 mm, or of at most 3.8 mm, or of at most 3.9 mm, or of at most 4.0 mm, or of at most 4.1 mm, or of at most 4.2 mm, or of at most 4.3 mm, or of at most 4.4 mm, or of at most 4.5 mm, or of at most 4.6 mm, or of at most 4.7 mm, or of at most 4.8 mm, or of at most 4.9 mm, or of at most 5.0 mm.

Preferably, the printing step (c) involves fused deposition modeling and the first pharmaceutical composition and the second pharmaceutical composition are deposited through separate extrusion dies (nozzles). Preferably, said separate extrusion dies have different dimensions, preferably a different diameter. The separate extrusion dies may also have a different shape according to the properties, e.g. viscosity, of the first and/or the second pharmaceutical composition.

In a preferred embodiment of the process according to the invention, in step (c) the first pharmaceutical composition and the second pharmaceutical composition are printed independently from each other. Preferably, the first pharmaceutical composition is printed at a different position within the pharmaceutical dosage form than the second pharmaceutical composition.

In a preferred embodiment of the process according to the invention, in step (c) the first pharmaceutical composition and the second pharmaceutical composition are printed in subsequent steps. Preferably, the first pharmaceutical composition is printed after the second pharmaceutical composition has been printed or the second pharmaceutical composition is printed after the first pharmaceutical composition has been printed. The time between the subsequent printing steps can range between a few seconds, hours or even days.

Preferably, all parts of the pharmaceutical dosage form comprising the first pharmaceutical composition are printed before the parts of the pharmaceutical dosage form comprising the second pharmaceutical composition are printed. For example, when the dosage from comprises only a small amount of the second pharmaceutical composition, this amount can be printed onto or into a structure which comprises or essentially consists of the first pharmaceutical composition. In a following step, said structure can be altered or the outer shape of the structure can be completed by further printing steps.

In another preferred embodiment of the process according to the invention, in step (c) the first pharmaceutical composition and the second pharmaceutical composition are printed simultaneously.

In a preferred embodiment of the process according to the invention, in printing step (c) according to the resolution of the printing device the pharmaceutical dosage form is printed by depositing individual three-dimensional microstructures having an individual volume of at most 1.5 mm³, or of at most 1.4 mm³, or of at most 1.3 mm³, or of at most 1.2 mm³, or of at most 1.1 mm³, or of at most 1.0 mm³, or of at most 0.9 mm³, or of at most 0.8 mm³, or of at most 0.7 mm³, or of at most 0.6 mm³, or of at most 0.5 mm³, or of at most 0.4 mm³, or of at most 0.3 mm³, or of at most 0.2 mm³, or of at most 0.1 mm³.

Preferably, printing the pharmaceutical dosage form involves fused deposition modeling and at least one extrusion die is heated to a temperature in the range of 40±5° C., or of 40±10° C., or of 40±15° C., or of 50±5° C., or of 50±10° C., or of 50±15° C., or of 50±20° C., or of 55±5° C., or of 55±15° C., or of 55±10° C., or of 55±15° C., or of 55±20° C., or 60±5° C., or 60±10° C., or 60±15° C., or 60±20° C., or 60±25° C., or 60±30° C., or 65±5° C., or 65±10° C., or 65±15° C., or 65±20° C., or 65±25° C., or 65±30° C., or 70±15° C., or 70±20° C., or 70±25° C., or 70±30° C., 80±15° C., or 80±20° C., or 80±25° C., or 80±30° C., 90±15° C., or 90±20° C., or 90±25° C., or 90±30° C., 100±5° C., or 100±10° C., 100±15° C., or 100±20° C., or 100±25° C., or 100±30° C., or 100±35° C., or 100±40° C., or 100±45° C., or 100±50° C., or 100±55° C., or 100±60° C., or 100±65° C., 110±5° C., or 110±10° C., 110±15° C., or 110±20° C., or 110±25° C., or 110±30° C., 120±5° C., or 120±10° C., 120±15° C., or 120±20° C., or 120±25° C., or 120±30° C., 130±5° C., or 130±10° C., 130±15° C., or 130±20° C., or 130±25° C., or 130±30° C., 140±5° C., or 140±10° C., 140±15° C., or 140±20° C., or 140±25° C., or 140±30° C., 150±5° C., or 150±10° C., 150±15° C., or 150±20° C., or 150±25° C., or 150±30° C., or 150±35° C., or 150±40° C., or 150±45° C., or 150±50° C., 160±5° C., or 160±10° C., 160±15° C., or 160±20° C., or 160±25° C., or 160±30° C., 100±5° C., or 180±10° C., 180±15° C., or 180±20° C., or 180±25° C., or 180±30° C., 190±5° C., or 190±10° C., 190±15° C., or 190±20° C., or 190±25° C., or 190±30° C., 100±5° C., or 200±10° C., 200±15° C., or 200±20° C., or 200±25° C., or 200±30° C.

In a preferred embodiment, the total amount of the first pharmacologically active ingredient is contained in the first pharmaceutical composition, whereas the total amount of the second pharmacologically active ingredient is contained in the second pharmaceutical composition.

In another preferred embodiment, the first pharmacologically active ingredient is divided in portions and one portion is contained in the first pharmaceutical composition whereas another portion is contained together with the second pharmacologically active ingredient in the second pharmaceutical composition.

In still another preferred embodiment, the second pharmacologically active ingredient is divided in portions and one portion is contained together with the first pharmacologically active ingredient in the first pharmaceutical composition whereas another portion is contained in the second pharmaceutical composition.

Preferably, the first and/or the second pharmaceutical composition comprises or essentially consists of an enteric material. Enteric materials are known to the skilled person. Preferably, the enteric material is selected from the group consisting of methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, and mixture thereof.

These pharmaceutical compositions preferably contain pharmaceutical excipients that are conventionally employed in the manufacture of pharmaceutical dosage forms, preferably in the course of three-dimensional printing technology, especially fused deposition modeling. The following preferred embodiments apply to the first pharmaceutical composition, the second pharmaceutical composition, the optional third pharmaceutical composition, the optional fourth pharmaceutical composition, and any another pharmaceutical composition (in the following commonly referred to as “pharmaceutical composition”), irrespective of whether they contain a pharmacologically active ingredient or not.

Preferably, the pharmaceutical composition comprises a plasticizer. Suitable plasticizers are known to the skilled person. Examples include but are not limited to polyethylene glycols, such as PEG 1500 or PEG 4000 or PEG 6000; citrates, phthalates, glycerin, sugar alcohols, various contents of copolymers (e.g. ethylene vinyl acetate (EVA)/vinyl acetate (VA)), and mixtures of any of the foregoing.

The content of plasticizer is preferably within the range of from 0.1 to 20 wt.-%, more preferably 5.0 to 17.5 wt.-%, still more preferably 7.5 to 15 wt.-%, relative to the total weight of the pharmaceutical composition.

Preferably, the pharmaceutical composition comprises one or more matrix polymers. Suitable matrix polymers are known to the skilled person. Examples include but are not limited to polylactic acid (PLA); cellulose ethers such as methylcellulose (MC), ethylcellulose (EC), hydroxypropylcellulose (HPC) and hydroxypropylmethylcellulose (HPMC); vinyl polymers such as polyvinylpyrrolidone (e.g. Kollidon® PF 12) or blends thereof such as polyvinyl acetate/polyvinylpyrrolidone (e.g. Kollidon® SR). Other suitable polymers include ethylene vinyl acetate copolymers (EVA), polyvinyl chloride, polyethylene terephthalate (PET), polyurethanes (PU), polyamides (PA), polyacrylates and mixtures of any of the foregoing.

The pharmaceutical composition may consist of one or more matrix polymers. The total content of matrix polymers is preferably within the range of from 5.0 to 95 wt.-%, more preferably 10 to 90 wt.-%, still more preferably 25 to 85 wt.-%, relative to the total weight of the pharmaceutical composition.

Representative pharmaceutical compositions that are useful for the purpose of the invention are compiled in the table here below. Composition 1 comprises a pharmacologically active ingredient and hence may serve as first pharmaceutical composition and second pharmaceutical composition, respectively, whereas Composition 2 comprises the same excipients in the same absolute amounts but no pharmacologically active ingredient and hence may serve as another pharmaceutical composition:

| | | Composition | | | |
|---|---|---|---|---|---|
| | | 1A | | Composition 2A | |
| | | weight | content | weight | content |
| ingredient | | [mg] | [wt.-%] | [mg] | [wt.-%] |
| pharma- | Tramadol HCl | 50 | 25 | — | — |
| cologically | | | | | |
| active ingredient | | | | | |
| plasticizer | PEG 4000 | 20 | 10 | 20 | 13.3 |
| matrix polymer | Ethylcellulose | 80 | 40 | 80 | 53.3 |
| matrix polymer | Polylactic acid | 50 | 25 | 50 | 33.3 |
| | | Composition | | | |
| | | 2A | | Composition 2B | |
| | | weight | content | weight | content |
| ingredient | | [mg] | [wt.-%] | [mg] | [wt.-%] |
| pharma- | Tramadol HCl | 0.5 | 0.1 | — | — |
| cologically | | | | | |
| active ingredient | | | | | |
| plasticizer | PEG 4000 | 49.5 | 9.9 | 49.5 | 9.9 |
| matrix polymer | Kollidon PF 12 | 450 | 90 | 450 | 90.1 |
| | | Composition | | | |
| | | 3A | | Composition 3B | |
| | | weight | content | weight | content |
| ingredient | | [mg] | [wt.-%] | [mg] | [wt.-%] |
| pharma- | Tramadol HCl | 204.08 | 40.8 | — | — |
| cologically | | | | | |
| active ingredient | | | | | |
| matrix polymer | Kollidon SR | 255.1 | 51.0 | 255.1 | 86.2 |
| matrix polymer | HPMC | 40.81 | 8.2 | 40.81 | 13.8 |

For filament preparation, a matrix polymer or a mixture of various matrix polymers, e.g. hydroxypropylcellulose (HPC), may be stored 24 h in oven at 40° C.; when required it may be mixed in a mortar with PEG 1500 or PEG 4000 (2%, 5%, 10% by weight calculated with respect to the dry polymer). Hot-melt extrusion (HME) may be carried out in a twin-screw extruder (Haake MiniLab II, Thermo Scientific, USA) equipped with an aluminum rod-shaped die (ø 2.00 mm). Extruded rods may be calibrated and rolled up on a spool.

Another aspect of the invention relates to a three-dimensionally printed pharmaceutical dosage form, which is preferably for oral administration, comprising
a first pharmacologically active ingredient; and
a second pharmacologically active ingredient;
   wherein the relative weight ratio of the first pharmacologically active ingredient to the second pharmacologically active ingredient in the pharmaceutical dosage form is within the range of from 10,000:1 to 1:1, preferably 10,000:1 to 20:1.

The pharmaceutical dosage form according to the invention is preferably obtainable by the process according to the invention as described above.

The pharmaceutical dosage form according to the invention has been manufactured by three-dimensional printing technology, preferably by fused-deposition modeling. Methods to distinguish such pharmaceutical dosage forms from other pharmaceutical dosage forms that have been manufactured by conventional techniques such as direct compression, extrusion, wet granulation, dry granulation, and the like are known to the skilled person and include but are not limited to microscopy and electron microscopy.

Preferably, the pharmaceutical dosage form comprises a first three-dimensionally printed pharmaceutical composition comprising the first pharmacologically active ingredient and a second three-dimensionally printed pharmaceutical composition comprising the second pharmacologically active ingredient, wherein the second pharmaceutical composition forms one voxel or at least two voxels which are spatially separated from one another.

For the purpose of the specification, a voxel (segment, element, subunit, part) may be the minimum three-dimensional microstructure than can be printed in accordance with the resolution of the printing device or an agglomerate of a multitude of such microstructures.

Preferably, the first three-dimensionally printed pharmaceutical composition forms a coherent mass. Preferably, the first three-dimensionally printed pharmaceutical composition forms a continuous phase within the pharmaceutical dosage form, wherein preferably voxels are embedded comprising the second three-dimensionally printed pharmaceutical composition, which in turn comprises the second pharmacologically active ingredient. In a preferred embodiment, the pharmaceutical dosage form essentially consists of the coherent mass which is formed by the first three-dimensionally printed pharmaceutical dosage form and optionally, the second three-dimensionally printed pharmaceutical composition.

Preferably, at least one voxel comprising the second three-dimensionally printed pharmaceutical composition has a volume of at most 1.5 mm³, or of at most 1.4 mm³, or of at most 1.3 mm³, or of at most 1.2 mm³, or of at most 1.1 mm³, or of at most 1.0 mm³, or of at most 0.9 mm³, or of at most 0.8 mm³, or of at most 0.7 mm³, or of at most 0.6 mm³, or of at most 0.5 mm³, or of at most 0.4 mm³, or of at most 0.3 mm³, or of at most 0.2 mm³, or of at most 0.1 mm³.

Preferably, at least one voxel comprising the second three-dimensionally printed pharmaceutical composition is embedded within the first three-dimensionally printed pharmaceutical composition. Preferably, the entire outer surface of the at least one voxel is surrounded by the first three-dimensionally printed pharmaceutical composition or only parts of the outer surface of the at least one voxel are surrounded by said first three-dimensionally printed pharmaceutical composition. Preferably, the at least one voxel forms a discontinuity in the coherent mass which is formed by the first three-dimensionally printed pharmaceutical composition.

In a preferred embodiment, the pharmaceutical dosage form comprises at least two voxels, which are each composed of the second three-dimensionally printed pharmaceutical composition, and which are spatially separated from one another. Preferably, the entire outer surface of each of the at least two voxels or of only one of the at least two voxels is surrounded by the first three-dimensionally printed pharmaceutical composition. Preferably, each of the at least two voxels forms a discontinuity within a coherent mass which is formed by the first three-dimensionally printed pharmaceutical composition.

In a preferred embodiment, the pharmaceutical dosage form comprises at least two voxels, which are each composed of the second three-dimensionally printed pharmaceutical composition, wherein the two voxels have the same volume. Preferably, each of the voxels has a volume of at most 1.5 mm³, or of at most 1.4 mm³, or of at most 1.3 mm³, or of at most 1.2 mm³, or of at most 1.1 mm³, or of at most 1.0 mm³, or of at most 0.9 mm³, or of at most 0.8 mm³, or of at most 0.7 mm³, or of at most 0.6 mm³, or of at most 0.5 mm³, or of at most 0.4 mm³, or of at most 0.3 mm³, or of at most 0.2 mm³, or of at most 0.1 mm³.

In another preferred embodiment, the pharmaceutical dosage form comprises at least two voxels, which are each composed of the second three-dimensionally printed pharmaceutical composition, wherein the two voxels have a different volume. Preferably, the total volume of all voxels, which are each composed of the second three-dimensionally printed pharmaceutical composition, comprised in the pharmaceutical dosage form is at most 3.0 mm³, 2.9 mm³, or at most 2.8 mm³, or at most 2.7 mm³, or at most 2.6 mm³, or at most 2.5 mm³, or at most 2.4 mm³, 2.3 mm³, or at most 2.2 mm³, or at most 2.1 mm³, or at most 2.0 mm³, or at most 1.9 mm³, or at most 1.8 mm³, or at most 1.7 mm³, or at most 1.6 mm³, or at most 1.5 mm³, or at most 1.4 mm³, or at most 1.3 mm³, or at most 1.2 mm³, or at most 1.1 mm³, or at most 1.0 mm³, or at most 0.9 mm³, or at most 0.8 mm³, or at most 0.7 mm³, or at most 0.6 mm³, or at most 0.5 mm³, or at most 0.4 mm³, or at most 0.3 mm³, or at most 0.2 mm³.

Preferably, the pharmaceutical dosage form has an outer surface and the at least two voxels, which are each composed of the second three-dimensionally printed pharmaceutical composition, have different shortest distances to said outer surface. After oral intake the pharmaceutical dosage form begins to dissolve in the gastric fluid, wherein the dissolution starts at the outer surface or exterior of the pharmaceutical dosage form and proceeds to the center of the pharmaceutical dosage form. During dissolution of the pharmaceutical dosage form the pharmacologically active ingredients comprised in the first and the second three-dimensionally printed pharmaceutical compositions are released from the pharmaceutical dosage form. Thus, the release profile of the pharmaceutical dosage form is a function of the length of the diffusion pathways from the exterior to the first pharmacologically active ingredient and to the second pharmacologically active ingredient, respectively. When the voxels, which are each composed of the second three-dimensionally printed pharmaceutical composition, have different shortest distances to the outer surface of the pharmaceutical dosage form, the pharmacologically active ingredient comprised in the voxels is released at different moments after oral intake of the pharmaceutical dosage form, wherein said moments depend on the individual distance of each voxel from the outer surface. By positioning the voxels at different shortest distances from the outer surface of the pharmaceutical dosage form it is possible to adapt the release profile of the second pharmacologically active ingredient from the pharmaceutical dosage form to a desired release profile. Such a release profile can depend on the properties of the drug (potency, efficacy) and/or on patients' needs.

Preferably, under in vitro conditions release of the second pharmacologically active ingredient from the two voxels does not commence simultaneously. Preferably, release of the second pharmacologically active ingredient comprised in one of the at least two voxels commences at least 5 minutes, or at least 10 minutes, or at least 20 minutes, or at least 30 minutes, or at least 40 minutes, or at least 50 minutes, or at least 60 minutes, or at least 70 minutes, or at least 80 minutes, or at least 90 minutes, or at least 100 minutes, or at least 110 minutes, or at least 120 minutes, or at least 130 minutes, or at least 140 minutes, or at least 150 minutes, or at least 160 minutes, or at least 170 minutes, or at least 180 minutes, or at least 190 minutes, or at least 200 minutes, or at least 210 minutes after release of the second pharmacologically active ingredient comprised in the other one of the at least two voxels commences.

Preferably, the at least two voxels have different shortest distances to the outer surface of the pharmaceutical dosage form and the pharmaceutical dosage form provides for a release profile that is n-modal, wherein n stands for the number of voxels which have different distances to the outer surface. For example, when a pharmaceutical dosage form comprises four voxels, which are each composed of the second three-dimensionally printed pharmaceutical composition, and three of the voxels are positioned at the same distance close to the outer surface and the other voxel is positioned at the center of the pharmaceutical dosage form, then there are two voxels which have different distances to the outer surface of the dosage from and the pharmaceutical dosage form will provide for a bimodal release profile of the second pharmacologically active ingredient. The three voxels which are positioned at the same distance to the outer surface will be dissolved first and release the second active ingredient at essentially the same moment, whereas the voxel which is positioned at the center of the pharmaceutical dosage form will be dissolved secondly and will release the second active ingredient at a later time than the three voxels.

Preferably, at least one of the two voxels, which are each composed of the second three-dimensionally printed pharmaceutical composition, is positioned on the outer surface of the pharmaceutical dosage form and at least one of said voxels is located in an intermediate layer of the pharmaceutical dosage form and/or is located in the core (inner body) of the pharmaceutical dosage form.

In a preferred embodiment, the first and the second pharmaceutical composition form different layers of the pharmaceutical dosage form, which are preferably adjacent and/or parallel to one another. In another preferred embodiment, the first and the second pharmaceutical composition together form a common layer of the pharmaceutical dosage form, wherein preferably the first pharmaceutical composition at least partially surrounds the second pharmaceutical composition within the plane of the layer.

Preferably, the pharmaceutical dosage form according to the invention under in vitro conditions provides release of the first pharmacologically active ingredient according to a first release kinetic and which provides release of the second pharmacologically active ingredient according to a second release kinetic, wherein said first release kinetic differs from said second release kinetic.

In a preferred embodiment, the pharmaceutical dosage form according to the invention under in vitro conditions provides immediate release of at least a portion of the first pharmacologically active ingredient and immediate release of at least a portion of the second pharmacologically active ingredient.

In another preferred embodiment, the pharmaceutical dosage form according to the invention under in vitro conditions provides immediate release of at least a portion of the first pharmacologically active ingredient and prolonged release of at least a portion of the second pharmacologically active ingredient.

In still another preferred embodiment, the pharmaceutical dosage form according to the invention under in vitro conditions provides prolonged release of at least a portion of the first pharmacologically active ingredient and prolonged release of at least a portion of the second pharmacologically active ingredient.

In yet another preferred embodiment, the pharmaceutical dosage form according to the invention under in vitro conditions provides prolonged release of at least a portion of the first pharmacologically active ingredient and immediate release of at least a portion of the second pharmacologically active ingredient.

For the purpose of the specification, immediate release preferably means that after 30 minutes under in vitro conditions the pharmaceutical dosage form has released at least 50 wt.-%, preferably at least 80 wt.-% of the total amount of the pharmacologically active ingredient that was originally contained in the pharmaceutical dosage form.

For the purpose of the specification, prolonged release preferably means that after 30 minutes under in vitro conditions the pharmaceutical dosage form has released not more than 50 wt.-%, preferably not more than 20 wt.-% of the total amount of the pharmacologically active ingredient that was originally contained in the pharmaceutical dosage form.

For the purpose of the specification, in vitro conditions preferably mean 900 mL artificial intestinal fluid (pH 6.8) in accordance with Ph. Eur. paddle method, at 50 rpm and 37° C.

Preferably, under in vitro conditions the release profile of the first pharmacologically active ingredient essentially corresponds to the release profile the second pharmacologically active ingredient. Preferably, at any point in time, under in vitro conditions the released amount of the first pharmacologically active ingredient differs from the released amount of the second pharmacologically active ingredient by absolutely not more than 10%, more preferably not more than 8%, still more preferably not more than 6%.

According to a preferred embodiment, the pharmaceutical dosage form according to the invention provides prolonged release of the first pharmacologically active ingredient and/or of the second pharmacologically active ingredient. Preferably, under in vitro condition in 900 mL artificial intestinal fluid (pH 6.8) in accordance with Ph. Eur. paddle method, at 50 rpm and 37° C., the pharmaceutical dosage form according to the invention exhibits a release profile independently of one another with regard to the first pharmacologically active ingredient and/or of the second pharmacologically active ingredient according to any of embodiments A′ to A⁸ as compiled in the table here below:

| | | A¹ | A² | A³ | A⁴ | A⁵ | A⁶ | A⁷ | A⁸ |
|---|---|---|---|---|---|---|---|---|---|
| 30 | min | ≥5% | ≥5% | ≥5% | ≥5% | ≥5% | ≥5% | ≥5% | ≥5% |
| 60 | min | ≥10% | ≥10% | ≥10% | ≥10% | ≥10% | ≥10% | ≥10% | ≥10% |
| 2 | h | 15-70% | 20-65% | 25-60% | 30-55% | 15-60% | 20-55% | 25-50% | 30-45% |
| 4 | h | ≤75 | ≤70 | ≤65 | ≤60 | 20-65% | 25-50% | 30-45% | 35-40% |
| 6 | h | ≤80% | ≤80% | ≤80% | ≤80% | 25-70% | 30-65% | 35-60% | 40-55% |
| 9 | h | ≥80% | ≥80% | ≥80% | ≥80% | ≤75 | ≤70 | ≤65 | ≤60 |
| 12 | h | ≥95% | ≥95% | ≥95% | ≥95% | ≤80% | ≤80% | ≤80% | ≤80% |
| 18 | h | ≥95% | ≥95% | ≥95% | ≥95% | ≥80% | ≥80% | ≥80% | ≥80% |
| 24 | h | ≥95% | ≥95% | ≥95% | ≥95% | ≥95% | ≥95% | ≥95% | ≥95% |

According to another preferred embodiment, the pharmaceutical dosage form according to the invention provides immediate release of the first pharmacologically active ingredient and/or of the second pharmacologically active ingredient. Preferably, under in vitro condition in 900 mL artificial intestinal fluid (pH 6.8) in accordance with Ph. Eur. paddle method, at 50 rpm and 37° C., the pharmaceutical dosage form according to the invention exhibits a release profile independently of one another with regard to the first pharmacologically active ingredient and/or of the second pharmacologically active ingredient according to any of embodiments B¹ to B⁸ as compiled in the table here below:

| | B¹ | B² | B³ | B⁴ | B⁵ | B⁶ | B⁷ | B⁸ |
|---|---|---|---|---|---|---|---|---|
| 30 min | ≥60% | ≥65% | ≥70% | ≥75% | ≥80% | ≥82.5% | ≥85% | ≥87.5% |
| 60 min | ≥75% | ≥80% | ≥82.5% | ≥85% | ≥87.5% | ≥90% | ≥92.5% | ≥95% |

According to a preferred embodiment, the pharmaceutical dosage form according to the invention provides an in vitro release profile of the first pharmacologically active ingredient in accordance with any of the above embodiments A¹ to A⁸ and independently of the second pharmacologically active ingredient in accordance with any of the above embodiments A¹ to A⁸.

According to another preferred embodiment, the pharmaceutical dosage form according to the invention provides an in vitro release profile of the first pharmacologically active ingredient in accordance with any of the above embodiments A¹ to A⁸ and independently of the second pharmacologically active ingredient in accordance with any of the above embodiments B¹ to B⁸.

According to still another preferred embodiment, the pharmaceutical dosage form according to the invention provides an in vitro release profile of the first pharmacologically active ingredient in accordance with any of the above embodiments B¹ to B⁸ and independently of the second pharmacologically active ingredient in accordance with any of the above embodiments A¹ to A⁸.

According to a preferred embodiment, the pharmaceutical dosage form according to the invention provides an in vitro release profile of the first pharmacologically active ingredient in accordance with any of the above embodiments B¹ to B⁸ and independently of the second pharmacologically active ingredient in accordance with any of the above embodiments B¹ to B⁸.

According to yet another preferred embodiment, the pharmaceutical dosage form according to the invention provides a release profile such that release of the first pharmacologically active ingredient commences after at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular essentially the total quantity of the second pharmacologically active ingredient, which was originally contained in the pharmaceutical dosage form, has been released already.

According to yet another preferred embodiment, the pharmaceutical dosage form according to the invention provides a release profile such that release of the second pharmacologically active ingredient commences after at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular essentially the total quantity of the first pharmacologically active ingredient, which was originally contained in the pharmaceutical dosage form, has been released already.

Preferably, the content of the first pharmacologically active ingredient is at least 1.0 wt.-%, or at least 2.0 wt.-%, or at least 3.0 wt.-%, or at least 4.0 wt.-%, or at least 5.0 wt.-%; more preferably at least 6.0 wt.-%, or at least 7.0 wt.-%, or at least 8.0 wt.-%, or at least 9.0 wt.-%, or at least 10 wt.-%; still more preferably at least 11 wt.-%, or at least 12 wt.-%, or at least 13 wt.-%, or at least 14 wt.-%, or at least 15 wt.-%; yet more preferably at least 16 wt.-%, or at least 17 wt.-%, or at least 18 wt.-%, or at least 19 wt.-%, or at least 20 wt.-%; even more preferably at least 21 wt.-%, or at least 22 wt.-%, or at least 23 wt.-%, or at least 24 wt.-%, or at least 25 wt.-%; most preferably at least 26 wt.-%, or at least 27 wt.-%, or at least 28 wt.-%, or at least 29 wt.-%, or at least 30 wt.-%; and in particular at least 31 wt.-%, or at least 32 wt.-%, or at least 33 wt.-%, or at least 34 wt.-%, or at least 35 wt.-%; relative to the total weight of the pharmaceutical dosage form.

Preferably, the content of the first pharmacologically active ingredient is at least 1 mg, or at least 2 mg, or at least 3 mg, or at least 4 mg, or at least 5 mg, or at least 6 mg, or at least 7 mg, or at least 8 mg, or at least 9 mg, or at least 10 mg; more preferably at least 11 mg, or at least 12 mg, or at least 13 mg, or at least 14 mg, or at least 15 mg, or at least 16 mg, or at least 17 mg, or at least 18 mg, or at least 19 mg, or at least 20 mg; still more preferably at least 21 mg, or at least 22 mg, or at least 23 mg, or at least 24 mg, or at least 25 mg, or at least 26 mg, or at least 27 mg, or at least 28 mg, or at least 29 mg, or at least 30 mg; yet more preferably at least 31 mg, or at least 32 mg, or at least 33 mg, or at least 34 mg, or at least 35 mg, or at least 36 mg, or at least 37 mg, or at least 38 mg, or at least 39 mg, or at least 40 mg; even more preferably at least 41 mg, or at least 42 mg, or at least 43 mg, or at least 44 mg, or at least 45 mg, or at least 46 mg, or at least 47 mg, or at least 48 mg, or at least 49 mg, or at least 50 mg; most preferably at least 51 mg, or at least 52 mg, or at least 53 mg, or at least 54 mg, or at least 55 mg, or at least 56 mg, or at least 57 mg, or at least 58 mg, or at least 59 mg, or at least 60 mg; and in particular at least 61 mg, or at least 62 mg, or at least 63 mg, or at least 64 mg, or at least 65 mg, or at least 66 mg, or at least 67 mg, or at least 68 mg, or at least 69 mg, or at least 70 mg, or at least 75 mg, or at least 80 mg, or at least 85 mg, or at least 90 mg, or at least 95 mg, or at least 100 mg, or at least 125 mg, or at least 150 mg, or at least 175 mg, or at least 200 mg, or at least 225 mg, or at least 250 mg, or at least 275 mg, or at least 300 mg, or at least 350 mg, or at least 400 mg, or at least 450 mg, or at least 500 mg.

Preferably, the content of the second pharmacologically active ingredient is at most 25 wt.-%, or at most 24 wt.-%, or at most 23 wt.-%, or at most 22 wt.-%, or at most 21 wt-%; more preferably at most 20 wt.-%, or at most 19 wt.-%, or at most 18 wt.-%, or at most 17 wt.-%, or at most 16 wt-%; still more preferably at most 15 wt.-%, or at most 14 wt.-%, or at most 13 wt.-%, or at most 12 wt.-%, or at most 11 wt-%; yet more preferably at most 10 wt.-%, or at most 9.0 wt.-%, or at most 8.0 wt.-%, or at most 7.0 wt.-%, or at most 6.0 wt-%; even more preferably at most 5.0 wt.-%, or at most 4.0 wt.-%, or at most 3.0 wt.-%, or at most 2.0 wt.-%, or at most 1.0 wt-%; most preferably at most 0.9 wt.-%, or at most 0.8 wt.-%, or at most 0.7 wt.-%, or at most 0.6 wt-%; and in particular at most 0.5 wt.-%, or at most 0.4 wt.-%, or at most 0.3 wt.-%, or at most 0.2 wt.-%, or at most 0.1 wt-%; relative to the total weight of the pharmaceutical dosage form.

Preferably, the content of the second pharmacologically active ingredient is at most 40 mg, or at most 39 mg, or at most 38 mg, or at most 37 mg, or at most 36 mg, or at most 35 mg, or at most 34 mg, or at most 33 mg, or at most 32 mg, or at most 31 mg; more preferably at most 30 mg, or at most 29 mg, or at most 28 mg, or at most 27 mg, or at most 26 mg, or at most 25 mg, or at most 24 mg, or at most 23 mg, or at most 22 mg, or at most 21 mg; still more preferably at most at most 20 mg, or at most 19 mg, or at most 18 mg, or at most 17 mg, or at most 16 mg, or at most 15 mg, or at most 14 mg, or at most 13 mg, or at most 12 mg, or at most 11 mg; yet more preferably at most 9.0 mg, or at most 8.0 mg, or at most 7.0 mg, or at most 6.0 mg, or at most 5.0 mg, or at most 4.0 mg, or at most 3.0 mg, or at most 2.0 mg, or at most 1.0 mg; even more preferably at most 900 μg, or at most 800 μg, or at most 700 μg, or at most 600 μg, or at most 500 μg, or at most 400 μg, or at most 300 μg, or at most 200 μg, or at most 200 μg; most preferably at most 90 μg, or at most 80 μg, or at most 70 μg, or at most 60 μg, or at most 50 μg, or at most 40 μg, or at most 30 μg, or at most 20 μg, or at most 10 μg;

and in particular at most 9.0 μg, or at most 8.0 μg, or at most 7.0 μg, or at most 6.0 μg, or at most 5.0 μg, or at most 4.0 μg, or at most 3.0 μg, or at most 2.0 μg, or at most 1.0 μg.

Preferably, the pharmaceutical dosage form according to the invention comprises a polymer matrix comprising a polymer selected from polylactic acid, cellulose ethers, vinyl polymers and mixtures thereof.

Preferably, the pharmaceutical dosage form according to the invention is a tablet for oral administration. Preferably, the tablet is round or oblong.

The total weight of the pharmaceutical dosage form according to the invention is not particularly limited. Preferably, the pharmaceutical dosage form according to the invention has a total weight within the range of 200±50 mg, or 200±200 mg, or 300±150 mg, or 400±200 mg, or 500±250 mg, or 600±300 mg, or 700±350 mg, or 800±400 mg, or 900±450 mg, or 2000±500 mg.

Preferably, the pharmaceutical dosage form according to the invention is monolithic. Preferably, the pharmaceutical dosage form according to the invention is not multiparticulate, e.g. dose not contain a multitude of hollow microspheres or a porous material. Preferably, the pharmaceutical dosage form according to the invention is a tablet.

The outer shape of the pharmaceutical dosage form according to the invention is not particularly limited. Preferably, the pharmaceutical dosage form according to the invention is
round or oblong and/or
flat or biconvex.

Preferably, the pharmaceutical dosage form according to the invention is for use in therapy, wherein the pharmaceutical dosage form is administered once daily or twice daily, preferably orally.

### BRIEF DESCRIPTION OF THE DRAWINGS

Preferred embodiments of the pharmaceutical dosage form according to the invention are also illustrated by FIGS. 1 to 3.

FIG. 1 schematically illustrates a pharmaceutical dosage form (1) according to the invention comprising body (2) that is composed of a first pharmaceutical composition comprising the first pharmacologically active ingredient and voxel (3) that is composed of a second pharmaceutical composition comprising the second pharmacologically active ingredient. Voxel (3) is positioned on the outer surface of the pharmaceutical dosage form (1). Voxel (3) may be the minimum three-dimensional microstructure than can be printed in accordance with the resolution of the printing device or an agglomerate of a multitude of such microstructures. Provided that due to the chosen ingredients of the first pharmaceutical composition and of the second pharmaceutical composition the release profile is a function of the length of the diffusion pathways from the exterior to the first pharmacologically active ingredient and to the second pharmacologically active ingredient, respectively, this embodiment will provide comparatively fast release of the second pharmacologically active ingredient.

FIG. 2 schematically illustrates a modification of the pharmaceutical dosage form (1) according to the invention. According to this embodiment, three voxels (3) are located in an intermediate layer of the pharmaceutical dosage form. Thus, material of body (2) must have been dissolved before release of the second pharmacologically active ingredient from voxels (3) may commence. This may be earlier the case for the two outer voxels (3a) and (3c) compared to the central voxel (3b).

FIG. 3 schematically illustrates another modification of the pharmaceutical dosage form (1) according to the invention. According to this embodiment, three voxels (3) are also located in an intermediate layer of the pharmaceutical dosage form, but in the center of the pharmaceutical dosage form (1).

FIG. 4 schematically illustrates a combination of the embodiments shown in FIGS. 1 and 3. Provided that due to the chosen ingredients of the first pharmaceutical composition and of the second pharmaceutical composition the release profile is a function of the length of the diffusion pathways from the exterior to the first pharmacologically active ingredient and to the second pharmacologically active ingredient, respectively, this embodiment will provide a bimodal release profile of the second pharmacologically active ingredient.

FIG. 5 schematically illustrates a modification of the pharmaceutical dosage form (1) according to the invention. According to this embodiment, two voxels (3) are located in different intermediate layers of the pharmaceutical dosage form.

FIG. 6 schematically illustrates a combination of the embodiments shown in FIGS. 1, 2 and 3. Provided that due to the chosen ingredients of the first pharmaceutical composition and of the second pharmaceutical composition the release profile is a function of the length of the diffusion pathways from the exterior to the first pharmacologically active ingredient and to the second pharmacologically active ingredient, respectively, this embodiment will provide a multimodal release profile of the second pharmacologically active ingredient.

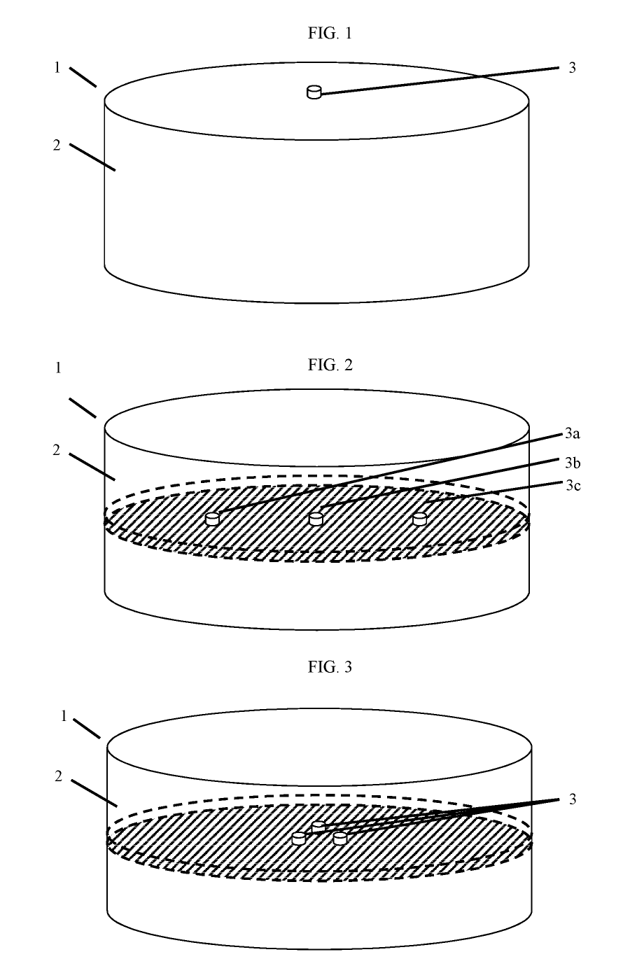

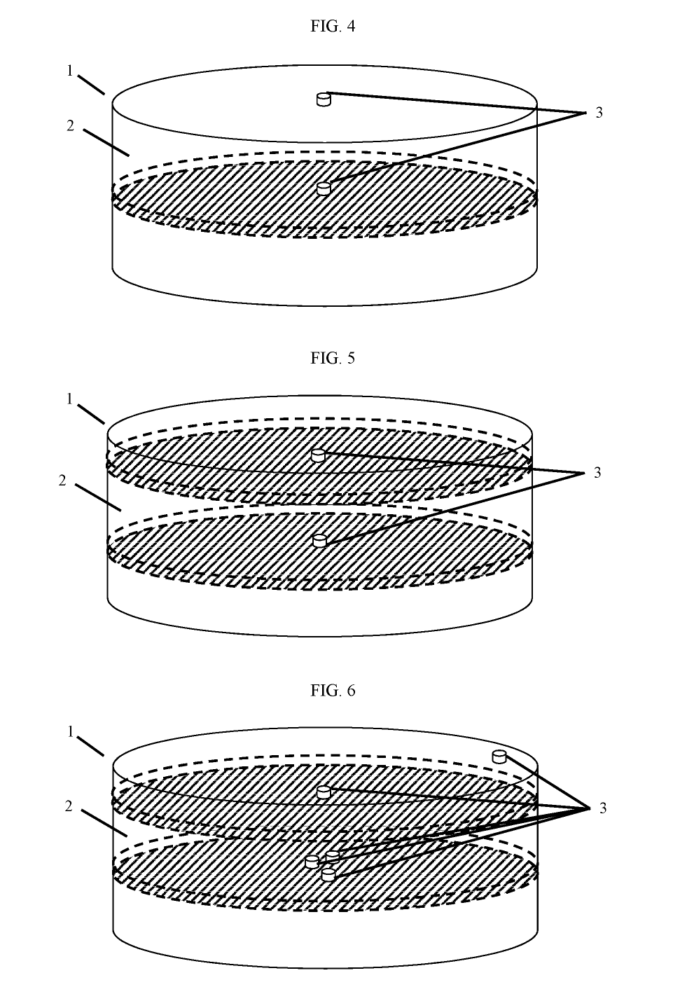

1. A three-dimensionally printed pharmaceutical dosage form for oral administration comprising
(i) a first pharmacologically active ingredient; and
(ii) a second pharmacologically active ingredient;
wherein the relative weight ratio of the first pharmacologically active ingredient to the second pharmacologically active ingredient in the pharmaceutical dosage form is within the range of from 10,000:1 to 20:1;
wherein the pharmaceutical dosage form comprises a first three-dimensionally printed pharmaceutical composition comprising the first pharmacologically active ingredient and a second three-dimensionally printed pharmaceutical composition comprising the second pharmacologically active ingredient; and
wherein the concentration of the first pharmacologically active ingredient in the first pharmaceutical composition is at least twice as high as the concentration of the second pharmacologically active ingredient in the second pharmaceutical composition.

2. The pharmaceutical dosage form according to claim 1, wherein the relative weight ratio of the first pharmacologically active ingredient to the second pharmacologically active ingredient in the pharmaceutical dosage form is within the range of from 10,000:1 to 250:1.

3. The pharmaceutical dosage form according to claim 1, wherein the second pharmaceutical composition forms one voxel or at least two voxels which are spatially separated from one another.

4. The pharmaceutical dosage form according to claim 1, wherein the first three-dimensionally printed pharmaceutical composition forms a coherent mass.

5. The pharmaceutical dosage form according to claim 3, wherein at least one voxel has a volume of at most 0.5 mm³.

6. The pharmaceutical dosage form according to claim 5, wherein the voxel has a volume of at most 0.1 mm3.

7. The pharmaceutical dosage form according to claim 3, wherein at least one voxel is embedded within the first three-dimensionally printed pharmaceutical composition.

8. The pharmaceutical dosage form according to claim 3, which comprises at least two voxels, which are each composed of the second three-dimensionally printed pharmaceutical composition, and which are spatially separated from one another.

9. The pharmaceutical dosage form according to claim 8, which has an outer surface and wherein the two voxels have different shortest distances to said outer surface.

10. The pharmaceutical dosage form according to claim 1, which provides under in vitro conditions release of the first pharmacologically active ingredient according to a first release kinetic and which provides release of the second pharmacologically active ingredient according to a second release kinetic, wherein said first release kinetic differs from said second release kinetic.

11. The pharmaceutical dosage form according to claim 1, which provides under in vitro conditions
(i) immediate release of at least a portion of the first pharmacologically active ingredient and immediate release of at least a portion of the second pharmacologically active ingredient; or
(ii) immediate release of at least a portion of the first pharmacologically active ingredient and prolonged release of at least a portion of the second pharmacologically active ingredient; or
(iii) prolonged release of at least a portion of the first pharmacologically active ingredient and immediate release of at least a portion of the second pharmacologically active ingredient; or
(iv) prolonged release of at least a portion of the first pharmacologically active ingredient and prolonged release of at least a portion of the second pharmacologically active ingredient.

12. The pharmaceutical dosage form according to claim 1, wherein the content of the first pharmacologically active ingredient is at least 10 wt.-%, relative to the total weight of the pharmaceutical dosage form.

13. The pharmaceutical dosage form according to claim 1, wherein the content of the second pharmacologically active ingredient is at most 10 wt.-%, relative to the total weight of the pharmaceutical dosage form.

14. The pharmaceutical dosage form according to claim 1, wherein the content of the first pharmacologically active ingredient is at least 25 mg.

15. A process for the preparation of a pharmaceutical dosage form according to claim 1, the process comprising the steps of
(a) providing a first three-dimensionally printable pharmaceutical composition comprising the first pharmacologically active ingredient;
(b) providing a second three-dimensionally printable pharmaceutical composition comprising the second pharmacologically active ingredient; and
(c) three-dimensionally printing the pharmaceutical dosage form from the first pharmaceutical composition and the second pharmaceutical composition.
